(19) United States
(12) Patent Application Publication
(10) Pub. No.: US 2023/0360808 A1
(43) Pub. Date: Nov. 9, 2023

(54) SERVER AND METHOD FOR INTEGRATED MANAGEMENT OF PERSONAL HEALTH RECORD FOR CHILD HAVING DEVELOPMENTAL DISORDER, AND RECORDING MEDIUM THEREOF

(71) Applicant: THE CATHOLIC UNIVERSITY OF KOREA INDUSTRY-ACADEMIC COOPERATION FOUNDATION, Seoul (KR)

(72) Inventors: Min-Hyeon PARK, Seoul (KR); Eun Kyung CHOI, Goyang-si (KR); Ha Yeon KIM, Seoul (KR); Ji Yea KIM, Incheon (KR)

(21) Appl. No.: 18/026,333

(22) PCT Filed: Sep. 17, 2021
(86) PCT No.: PCT/KR2021/012846

§ 371 Date: Mar. 14, 2023

(30) Foreign Application Priority Data
Sep. 21, 2020 (KR) 10-2020-0121516
Jul. 13, 2021 (KR) 10-2021-0091858

(51) Int. Cl. G16H 20/10; G16H 80/00; G16H 10/60; G16H 30/20

(52) U.S. Cl. CPC G16H 80/00; G16H 10/60; G16H 20/10; G16H 30/20

(57) ABSTRACT

An apparatus and method, and a recording medium thereof, wherein the apparatus receives image data, recognizes, from the received image data, information selected from among image information and text information, controls one of the recognized image information and text information to be discriminately stored in a predetermined data format according to patient information, clinical information, and post-treatment change progress information, and displays, according to a request, a personal health record including the stored patient information, clinical information, and post-treatment change progress information.

### TECHNICAL FIELD

The present invention provides a server and method for integratedly managing personal health records for children with developmental disabilities and a recording medium thereof.

### BACKGROUND ART

Personal health record (PHR) is a health management technology and encompasses biometric signal monitoring technology, home healthcare/home care service technology, mobile/U healthcare service technology, and patient management monitoring technology. Personal health record platform technology is divided into electronic medical record (EMR) system technology, data storage and integration technology, data exchange and sharing technology, and personal health information protection technology.

Conventional personal health records are difficult to manage because it is difficult to capture and easily create digital data of existing checkup records or prescriptions. Further, conventional personal health records have low utilization because they cannot perform chatting or provide medication information in connection with personal health records.

Further, conventional personal health records cannot detect and automatically indicate problematic behavior/medication status of children with developmental disabilities, and cannot provide a medication compliance enhancement program or side effect management system for children with developmental disabilities.

### DETAILED DESCRIPTION OF THE INVENTION\

### Technical Problem

An embodiment may provide a server, method and recording medium for easily storing and managing existing checkup records or prescriptions as personal health records.

Another embodiment may provide a server and method for performing chatting or providing medication information in association with stored and managed personal health records, and a recording medium thereof.

Another embodiment may provide a server and method for detecting problematic behavior/medication status of children with developmental disabilities and providing an automatic alarm, and a recording medium thereof.

Another embodiment may provide a server and method for providing a medication compliance enhancement program or side effect management system for children with developmental disabilities, and a recording medium thereof.

### Technical Solution

The disclosure includes a device and method, and a recording medium thereof, comprising recognizing one of image information and text information from received image data, controlling to divide and store one of the recognized image information and text information into patient information and clinical information in a predetermined data format, and requesting a personal health record including the stored patient information, clinical information, and change trend information according to treatment.

An embodiment provides a server for integratedly managing personal health records for children with developmental disabilities.

The server comprises a communication unit communicating with another server or a terminal through a network and receiving image data from the other server or the terminal, a storage unit storing a personal health record, including patient information and clinical information, and an input unit receiving image data. The server comprises a controller recognizing one of image information and text information from the received image data, dividing and storing, in the storage unit, one of the recognized image information and the text information into the patient information and the clinical information in a predetermined data format and an output unit displaying the personal health record including the patient information and the clinical information stored in the storage unit, according to a request.

Another embodiment provides a method for integratedly managing personal health records, including clinical information for children with developmental disabilities and changing trend information according to treatment. The method comprises receiving image data, recognizing one of image information or text information from the received image data and controls to divide and store one of the recognized image information and the text information into the patient information and the clinical information in a predetermined data format, and displaying the personal health record including the stored patient information, the clinical information, and the change trend information according to treatment according to a request.

### Advantageous Effects

The server, method, and recording medium for integratedly managing personal health records for child with developmental disabilities according to embodiments may easily store and manage existing test records or prescriptions as personal health records.

The server, method, and recording medium for integratedly managing personal health records for child with developmental disabilities according to embodiments may perform chatting or provide medication information in association with stored and managed personal health records.

The server, method, and recording medium for integratedly managing personal health records for child with developmental disabilities according to embodiments may detect problematic behavior/medication status of children with developmental disabilities and provide an automatic alarm.

The server, method, and recording medium for integratedly managing personal health records for child with developmental disabilities according to embodiments may provide a medication compliance enhancement program or side effect management system for children with developmental disabilities.

### BRIEF DESCRIPTION OF THE DRAWINGS

FIG. 1 is a view illustrating a system for integratedly managing personal health records for children with developmental disabilities according to an embodiment of the present invention.

FIG. 2A is a block diagram illustrating a server for integratedly managing personal health records for children with developmental disabilities, included in the system of FIG. 1.

FIGS. 2B and 2E illustrate screens on which an output unit provides a description for each test result and answers to questions about the test.

FIG. 3A is a view illustrating a configuration of an example of a controller of the server of FIG. 2A.

FIG. 3B is a view illustrating a configuration of another example of a controller of the server of FIG. 2A.

FIG. 4 illustrates an example of image data.

FIG. 5 is a view illustrating a process of recognizing image information and text information from the image data of FIG. 4 and separating and storing them in a predetermined data format by the image recognition unit and the OCR unit of FIG. 3A.

FIGS. 6A to 6C illustrate examples of test results newly stored.

FIG. 7 illustrates an example of an automatic alarm message.

FIG. 8 is a flowchart illustrating a method for integratedly managing personal health records for children with developmental disabilities according to another embodiment.

### MODE FOR CARRYING OUT THE INVENTION

Hereinafter, exemplary embodiments of the inventive concept will be described in detail with reference to the accompanying drawings. However, the inventive concept may be modified in various ways, and should not be construed as limited to the embodiments set forth herein. Like reference denotations may be used to refer to the same or similar elements throughout the specification and the drawings. However, the present invention may be implemented in other various forms and is not limited to the embodiments set forth herein. For clarity of the disclosure, irrelevant parts are removed from the drawings, and similar reference denotations are used to refer to similar elements throughout the specification.

In embodiments of the present invention, when an element is “connected” with another element, the element may be “directly connected” with the other element, or the element may be “electrically connected” with the other element via an intervening element. When an element “comprises” or “includes” another element, the element may further include, but rather than exclude, the other element, and the terms “comprise” and “include” should be appreciated as not excluding the possibility of the presence or adding one or more features, numbers, steps, operations, elements, parts, or combinations thereof.

When the measurement of an element is modified by the term “about” or “substantially,” if a product or material tolerance is provided for the element, the term “about” or “substantially” is used to indicate that the element has the same or a close value to the measurement and is used for a better understanding of the present invention or for preventing any unscrupulous infringement of the disclosure where the exact or absolute numbers are mentioned. As used herein, “step of” A or “step A-ing” does not necessarily mean that the step is one for A.

As used herein, the term “part” may mean a unit or device implemented in hardware, software, or a combination thereof. One unit may be implemented with two or more hardware devices or components, or two or more units may be implemented in a single hardware device or component.

As used herein, some of the operations or functions described to be performed by a terminal or device may be, instead of the terminal or device, performed by a server connected with the terminal or device. Likewise, some of the operations or functions described to be performed by a server may be performed by a terminal or device connected with the server, instead of the server.

Hereinafter, embodiments of the present invention are described in detail with reference to the accompanying drawings.

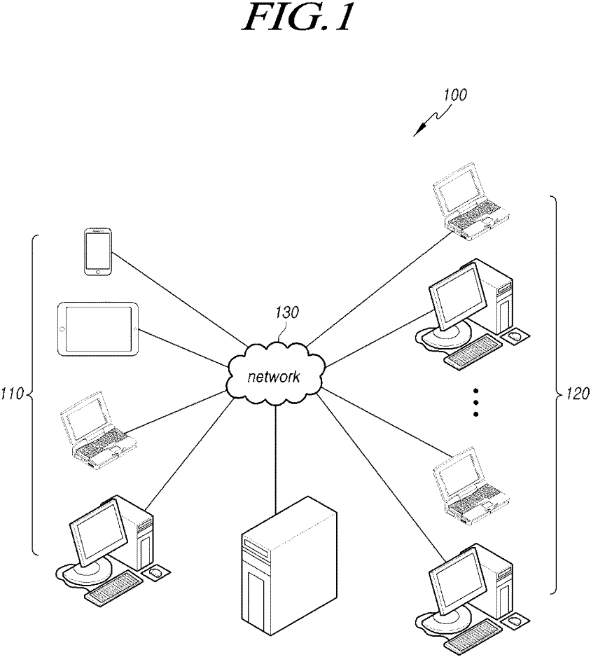

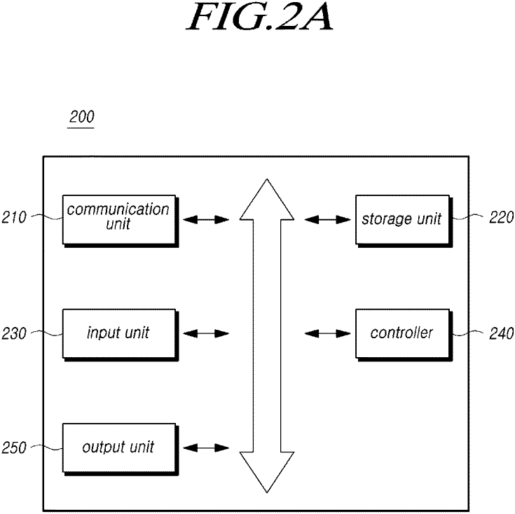

FIG. 1 is a view illustrating a system for integratedly managing personal health records for children with developmental disabilities according to an embodiment of the present invention.

Referring to FIG. 1, a system 100 for integratedly managing personal health records for child with developmental disabilities may include a patient terminal 110, a server 200 for integratedly managing personal health records for children with developmental disabilities, and a medical staff terminal 120.

The components of the system 1 are connected together via a network 130. For example, as shown in FIG. 1, a patient terminal 110 may be connected with the server 200 for integratedly managing personal health records for children with developmental disabilities, through a network 130. The server 200 for integratedly managing personal health records for children with developmental disabilities may be connected with the patient terminal 110 and the medical staff terminal 120 through the network 130. Further, the medical staff terminal 120 may be connected with the server 200 for integratedly managing personal health records for children with developmental disabilities through the network 130.

Here, the term “network” means a connecting structure to enable information exchange between nodes, such as a plurality of terminals and servers. Examples of such networks may include, but are not limited to, a radio frequency (RF) network, a 3rd Generation Partnership Project (3GPP) network, a Long Term Evolution (LTE) network, a Long Term Evolution-Advanced (LTE-A) network, a 5th Generation Partnership Project (5GPP) network, a World Interoperability for Microwave Access (WIMAX) network, an Internet network, a Local Area Network (LAN) network, a Wireless LAN network, a Wide Area Network (WAN) network, a Personal Area Network (PAN) network, a Bluetooth network, a satellite broadcast network, an analog broadcast network, and a Digital Multimedia Broadcasting (DMB) network.

As used herein, the singular forms “a,” “an,” and “the” are intended to include the plural forms as well, unless the context clearly indicates otherwise. According to embodiments, a plurality of components of the same type may be a single component of the type, and one component may add one or more components of the same type.

The patient terminal 110 may be a terminal of a child with developmental disabilities or a patient who requests treatment using a personal health record-related webpage, app page, program or application including change trend information according to treatment and clinical information about the child with developmental disabilities or his/her caregiver. In this case, a patient terminal 110 may be a terminal that transmits the patient’s medical records to the server 200 for integratedly managing personal health records, including change trend information according to treatment and clinical information about children with developmental disabilities, including text, images, and videos. Further, the patient terminal 110 may be a terminal that receives feedback for a diagnosis request from the server 200 for integratedly managing personal health records for children with developmental disabilities.

The patient terminal 110 may be implemented as a computer capable of accessing a remote server or terminal via the network. Here, the computer may be, e.g., a navigation or web browser-equipped laptop or desktop computer. At least one diagnosis request terminal 100 may be implemented as a terminal capable of accessing a remote server or terminal via the network. The patient terminal 110 may be, e.g., a portable mobile wireless communication device examples of which may include navigation devices, a Personal Communication System (PCS), Global System for Mobile communications (GSM), Personal Digital Cellular (PDC), Personal Handyphone System (PHS), Personal Digital Assistant (PDA), International Mobile Telecommunication (IMT)-2000, Code Division Multiple Access (CDMA)-2000, W-Code Division Multiple Access (W-CDMA), Wireless Broadband Internet (WiBro) terminal, a smartphone, a smart pad, tablet PC, or any other various types of handheld wireless communication devices.

The server 200 for integratedly managing personal health records for children with developmental disabilities may be a server that provides a personal health record-related webpage, app page, program or application including change trend information according to treatment and clinical information about the child with developmental disabilities.

The server 200 for integratedly managing personal health records for children with developmental disabilities may be implemented as a computer capable of accessing a remote server or terminal via the network. Here, the computer may be, e.g., a navigation or web browser-equipped laptop or desktop computer.

The medical staff terminal 120 may be a terminal of medical staff, such as a diagnostician or doctor or play therapist using a personal health record-related webpage, app page, program or application including change trend information according to treatment and clinical information about the child with developmental disabilities. The medical staff terminal 120 may be a terminal that receives the medical record data, which has been received from the patient terminal 110, from the server 200 for integratedly managing personal health records for children with developmental disabilities and transmits, e.g., opinions on the received medical record data to the server 200 for integratedly managing personal health records for children with developmental disabilities. Further, the medical staff terminal 120 may be a terminal that receives results of inquiries until big data is assimilated by artificial intelligence in the server 200 for integratedly managing personal health records for children with developmental disabilities and feeds errors back to the server 200 for integratedly managing personal health records for children with developmental disabilities.

The medical staff terminal 120 may be implemented as a computer capable of accessing a remote server or terminal via the network. Here, the computer may be, e.g., a navigation or web browser-equipped laptop or desktop computer. The medical staff terminal 120 may be implemented as a terminal capable of accessing a remote server or terminal via the network. The medical staff terminal 120 may be, e.g., a portable mobile wireless communication device examples of which may include navigation devices, a Personal Communication System (PCS), Global System for Mobile communications (GSM), Personal Digital Cellular (PDC), Personal Handyphone System (PHS), Personal Digital Assistant (PDA), International Mobile Telecommunication (IMT)-2000, Code Division Multiple Access (CDMA)-2000, W-Code Division Multiple Access (W-CDMA), Wireless Broadband Internet (WiBro) terminal, a smartphone, a smartpad, tablet PC, or any other various types of handheld wireless communication devices.

When another server nutritional supplement which operates in conjunction with the server 200 for integratedly managing personal health records for children with developmental disabilities according to an embodiment of the present invention transmits, to the patient terminal 110 and the medical staff terminal 120, e.g., an application, program, app page, or webpage related to change trend information according to treatment and clinical information about the child with developmental disabilities, the patient terminal 110 and the medical staff terminal 120 may install the personal health record-related application, program, app page, or webpage including the change trend information according to treatment and the clinical information about the child with developmental disabilities. Further, a service program may be driven or run on the patient terminal 110 and the medical staff terminal 120 using a script executed on a web browser. Here, the web browser may be a program or application that enables use of world wide web (WWW) services or that receives and shows hypertext written in the hypertext mark-up language (HTML), and the web browser may include, e.g., Netscape, Explorer, or Chrome. The term “application” may mean an application executed on the terminal, and the application may include, e.g., an app running on a mobile terminal, e.g., a smartphone.

Electronic medical record system technology, data storage and integration technology, data exchange and sharing technology, and personal health information protection technology may be applied to the server 200 for integratedly managing personal health records to build a personal health record platform. The electronic medical record system technology is an electronic medical record that preserves all medical records as electronic documents, and the data storage and integration technology is a technology that stores and integrates personal health record data to provide personal health information and services. The data exchange and sharing technology is personal health record data to provide personal health information and services, and the personal health information protection technology is a technology to protect personal health record data to provide personal health information and services.

Although the following description focuses primarily on the server 200 for integratedly managing personal health records, to which the data storage and integration technology and data exchange and sharing technology and personal health information protection technology are applied, the present invention is not limited thereto.

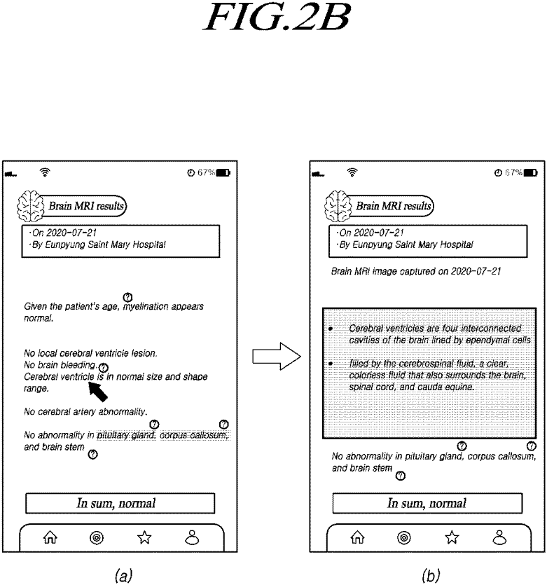

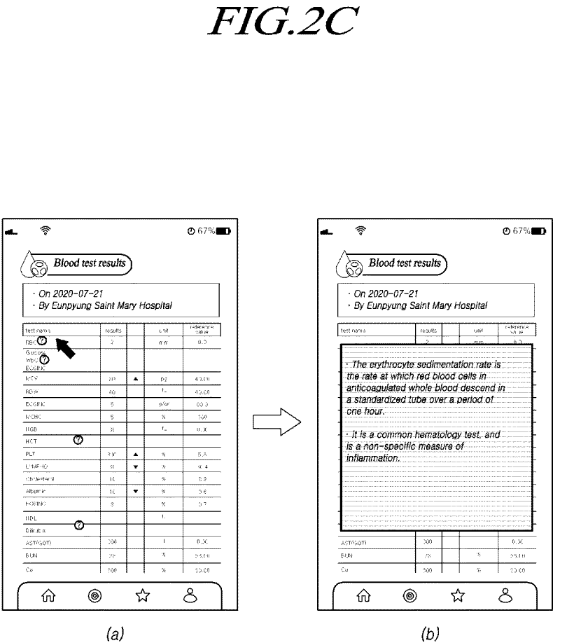

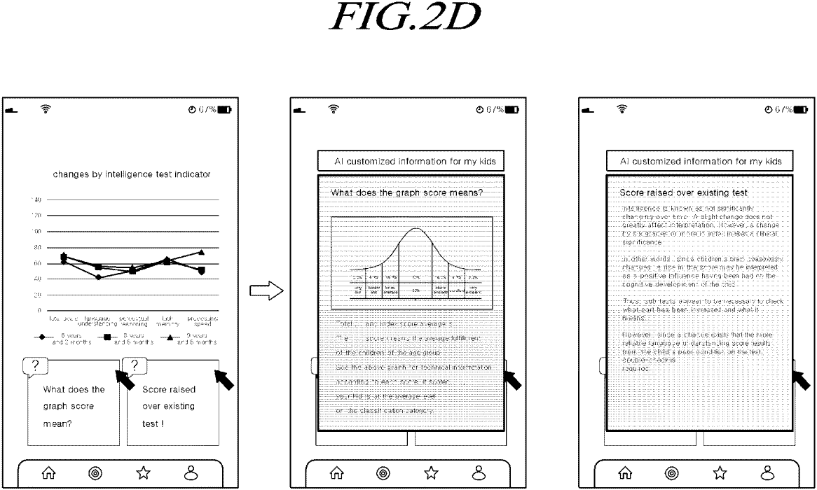

FIG. 2A is a block diagram illustrating a server for integratedly managing personal health records for children with developmental disabilities, including in the system of FIG. 1.

Referring to FIG. 2A, the server 200 for integratedly managing personal health records includes a communication unit 210 communicating with another server or terminal through a network 130, a storage unit 220 storing data, an input unit 230 inputting data, an output unit 250 displaying images or outputting displayed data to the outside, and a controller 240 controlling them.

The communication unit 210 may receive image data from another server or a UE. The other server (not shown) may operate in conjunction with the server 200 for integratedly managing personal health records as according to an embodiment. The UE may be the patient terminal 110 and/or medical staff terminal 120 described in connection with FIG. 1 or be a third UE (not shown) separate therefrom.

The storage unit 220 may be a common storage device. In particular, the storage unit 220 may store thepersonal health records including patient information, clinical information, and change trend information according to treatment.

In this case, the personal health record (PHR) means any data or information related to personal health, such as an electronic health record (HER) and smart health data.

The personal health record may include patient information, daily management information (e.g., medication management information, side effects management information, or treatment log information), test report in Table 1, questionnaire and psychological scale in Table 2, FAQ, etc. Further, the personal health record may include information obtained by the operation of each component of the controller 240 to be described below.

TABLE 1

Test report

| | |
|---|---|
| A | Brain magnetic resonance imaging of the target child |
| B | Blood and urine test results |
| C | Infant and young children clinical test results |
| C | Korean Wechsler Preschool Primary Scale of Intelligence (K-WIPPISI) |
| | - Korean Wechsler Intelligence Scale for Children (K-WISC) |
| | - Korean Wechsler Adult Intelligence Scale (K-WAIS) |
| | - Kaufman Assessment Battery for Children (K-ABC) |
| | - Korean-Leiter-Revised (K-Leiter-R) |
| | - The Beery-Buktenica Developmental Test of Visual-Motor Integration (Breery VMI-6) |
| | - Denver Developmental Screening Test-II (DDST-2) |
| | - Korean-Bayley Scales of Infant Development-III (K-Bayley-III) |
| | - Psycho Educational Profile (PEP) |
| | - Social Maturity Scale (SMS) |
| | - Korean-Vineland Adaptive Behavior Scales Second Edition (K-Vineland-II) |
| | - Autism Diagnostic Observation Schedule (ADOS) |
| | - Autism Diagnostic Interview – Revised (ADI-R) |
| | - Childhood Autism Rating Scale (CARS) |
| | - Korean-Childhood Autism Rating Scale 2 (K-CARS2) |
| | - Children’s Color Trails Test (CCTT) |
| | - color and word test children’s version (STROOP) |
| | - Wisconsin Card Sorting Test (WCST) |
| | - Advances Test of Attention (ATA) |
| | - Comprehensive Attention Test (CAT) |
| | - Comprehensive Learning Test (CLT) |
| | - Minnesota Multiphasic Personality Inventory-II (MMPI-II) |
| | - Minnesota Multiphasic Personality Inventory-adolescent (MMPI-A) |
| | - Temperament and Character Inventory; JTCI for 3-6 years old children / JTCI for 7-11 years old children / JTCI for 12-18 years old children) |
| | - Child Behavior Checklist (CBCL) |
| | - Sequenced Language Scale for Infants (SELSI) |
| | - Preschool Receptive-Expressive Language Scale (PRES) |
| | - Receptive and Expressive vocabulary Test (REVT) |
| | - Assessment of Phonology & Articulation for Children (APAC) |
| | - Urimal Test of Articulation and Phonology (UTAP) |
| | - Peabody Developmental Motor Scales (PDMS-2) |
| | - Alberta Infant Motor Scales |
| | - Early Coping Inventory |
| | - Gross Motor Function Measure (GMFM) |
| | - Developmental Test of Visual Perception II (DTVP-2) |
| | - Motor-Free Visual Perception Test (MVPT-2) |
| | - Erhardt Developmental Prehension Assessment (EDPA) |
| | - Behavior Rating Inventory of Executive Function-Preschool Version (BRIEF-P) |

The test report may be translated in terms that may easily be understood by the caregivers and provide a detailed description.

TABLE 2

| | |
|---|---|
| | Questionnaires and psychological scales |
| Caregiver-related | Parenting Stress index (PSI) |
| Caregiver-related | Korean version of Beck Depression index (K-BDI) |
| Related to the subject’s problem behavior | Korean version of The Child Development Inventory (K-CDI) |
| Related to the subject’s problem behavior | Kovacs’ depression scale (CDI) |
| Related to the subject’s problem behavior | Revised Children’s Manifest Anxiety Scale (RMCAS) |
| Related to the subject’s problem behavior | Korean version of Beck Anxiety Inventory (K-BAI) |
| Related to the subject’s problem behavior | ADHD Rating Scale(K-ADHD-RS) |
| Related to the subject’s problem behavior | |

Personal health records may be divided into personal health records for patients and personal health records for medical staff. As described below, the personal health records for patients may be customized information for caregivers and customized treatment program recommendation information. The personal health records for medical staff may be medical information through participation of medical staff and information for identifying the patient’s life log. The input unit 230 may be a general input device capable of inputting audio, an image, or text, such as a keyboard, a mouse, a camera, a recorder, a wearable device, and various sensors. The input unit 230 may receive image data through, e.g., a camera device.

The controller 240 may recognize one of image information and text information from the received image data, divide the recognized one of the image information and text information into patient information and clinical information or patient information and clinical information and change trend information according to treatment, in a predetermined data format, and store them in the storage unit 220.

The controller 240 may analyze the personal health record profile of the child, matches the categories of users with similar profiles to the corresponding interpreted information (psychological assessment information database), searches for customized information based thereon, and then provide per-profile customized information which is customized information for the caregiver. Thus, the controller 240 may analyze the personal health record stored by the caregiver, extract the child’s developmental information, and provide service or customized treatment program recommendation information that may recommend useful developmental stimuli according thereto.

The controller 240 may recommend an evidence-based treatment module when a clinically significant functional decline is observed in the developmental assessment. For example, the controller 240 may recommend evidence-based treatment modules according to developmental and emotional states through artificial intelligence. Evidence-based treatment modules refer to treatments whose effects have been found through well-designed studies, and may help establish a more accurate treatment plan.

The controller 240 may build big data through matching between the knowledge base (clinician’s interpretation) and the database of the storage unit 220 and form and enhance a specific algorithm.

The controller 240 may integratedly recognize tests of hospitals, which are created in different units and expression schemes even for the same test. The controller 240 may make be compatible with data and integratedly recognize the results of the same test executed in several development centers as well as from hospitals, particularly when the tests are made for children with developmental disabilities.

The output unit 250 may display the personal health information including the patient information and clinical information and the change trend information stored in the storage unit 220, according to a request.

The image data may be classified into structured semi structured and unstructured data depending on the structured degree.

The unstructured data are items that are difficult to define due to their irregular form and may generally include text and images. In the medical field, since most text data may be analyzed as semi-structured, only image or video data may be considered as unstructured. The unstructured data may include, e.g., various types of video data, such as coronary angiography (CAG) and various sonograms, and image data, such as computed tomography (CT), magnetic resonance imaging (MRI), and electrocardiogram (ECG). Such unstructured data is also accessed with key words, tags, or metadata, and is classified and patterned. When classification of new unstructured data is requested later, the data may be processed into a record format and provided together with associated data.

The image data may be one of image data of test data prepared in a specific data format, prescription image data, and a medical report according to the type of information.

In general, for tests adopted worldwide, test results are written differently per test organization. For example, test results may be expressed as standard scores (e.g., IQ, t-score, z-score), %ile (percentile), high, medium, and low, and excellent/poor. Correspondingly, the output unit 250 OCRs and digitize the test result and outputs unified data by making test results from several hospitals and test organizations compatible.

Because the test terminology is difficult to understand, it may be nearly impossible for the caregiver to properly judge and utilize the test details only with the results. Accordingly, output unit 250 outputs the test report that may translate into terms that may easily be understood by the caregivers of the children with developmental disabilities and provide a detailed description.

FIGS. 2B and 2C illustrate screens on which an output unit 250 provides a description for each test result and answers to questions about the test.

Referring to FIGS. 2B and 2C, when displaying an description of each test result, the output unit 250 may attach tags to difficult terms or items that may be curious about the test. When the user selects one of the tags, the output unit 250 may blur the test result and provide a detailed description of the esoteric term or questions about the test with the selected tag, linked to an Internet search information or medical terminology dictionary.

For example, as shown in FIG. 2B, when the user clicks the tag attached to the ventricle in the test result, the output unit 250 may provide a detailed description of the ventricle by linking to a medical terminology dictionary.

As another example, as shown in FIG. 2C, when the user clicks ESR, which is one of the test names in the test results, the output unit 250 may provide a detailed description of the ESR by linking to a medical terminology dictionary.

Further, the output unit 250 outputs a test record to be able to observe follow-ups and changes for the individual’s annual test record. Further, the output unit 250 may output follow-up observation results of changes by indicator for each subtest and changes by developmental period for one test, thus output customized information.

FIG. 2D sequentially illustrates screens where the output unit 250 outputs changes for each intelligence test indicator and AI-based customized information for one test. For example, the controller 240 controls the output unit 250 to output user-customized information through profile analysis of various information, such as profile analysis of intelligence test development information and/or profile analysis of temperament and personality information, and profile analysis of emotional state information. Thus, the server 200 may provide user-customized information through profile analysis of each test.

Further, the output unit 250 may convert the comparable top items into a unified unit value (percentile %), integrate them as a “comprehensive development report”, and visualize it as a radial graph. Thus, the user or medical staff may assess the overall development status at a glance. FIG. 2E illustrates a part of the comprehensive development report output by the output unit.

The server 200 may provide customized welfare information according to expected diagnosis and disabilities according to the output of the comprehensive development report. For example, the server 200 may provide legal information, such as the Welfare Act for Persons with Disabilities from the government, public institutions, and private organizations, and support information, such as national support/local government support or private organization support. The user may access the server 200 and utilize customized welfare information, such as the above-described legal information or support information.

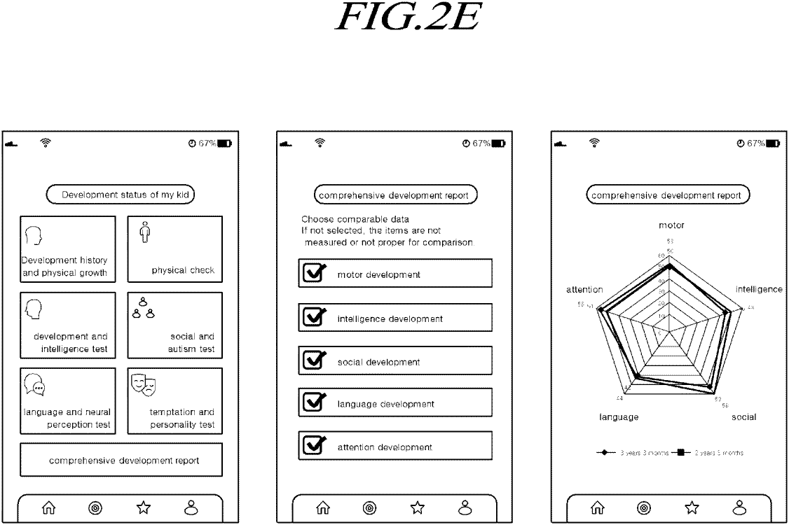

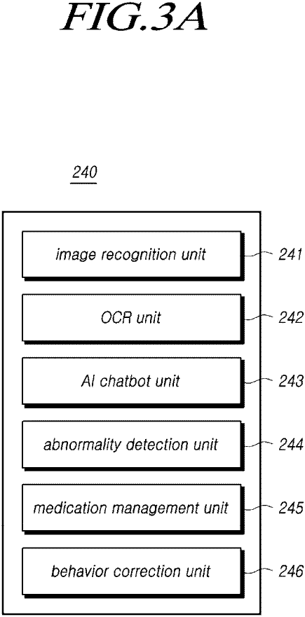

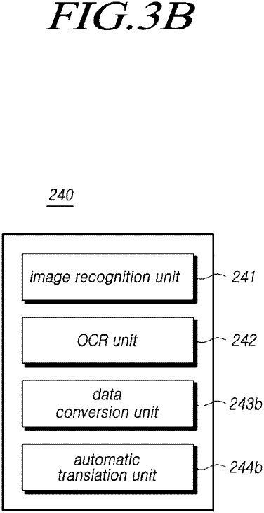

FIG. 3A is a view illustrating a configuration of a controller of the server of FIG. 2A.

Referring to FIG. 3A, the controller 240 may include an image recognition unit 241 that recognizes image information from one of the image data of test data, prescription image data, and medical report and an optical character recognition (OCR) unit 242 that recognizes text information from one of the image data of test data, prescription image data, and medical report.

The controller 240 may be classified one of the image information and text information recognized from one of the image data of test data, prescription image data, and medical report created in a specific data format into patient information and clinical information and change trend information according to treatment according to a predetermined data format and store them in the storage unit 220.

The image recognition unit 241 may recognize image information from one of the image data of test data, prescription image data, and medical report and be classified them into patient information and clinical information and change trend information according to treatment according to a predetermined data format and store them in the storage unit 220.

The image recognition unit 241 may recognize the image information from the input image data using deep learning technology which uses the artificial neural network or general image recognition technology, grasp the meaning of the recognized image information, be classified the recognized image information according to a predetermined data format 316 depending on the type of the recognized image information and store them in the storage unit 220.

The OCR unit 242 may use a pipeline that sequentially performs binarization, word segmentation, and character segmentation on the character image included in the image data using, e.g., optical character recognition technology, and then recognizes individual characters.

The OCR unit 242 may also use the general character recognition technology and may use deep learning technology which uses the artificial neural network to recognize the text information from the input image data, grasp the meaning of the text information and separate the personal health record according to the type of each piece of information according to a predetermined data format, and store them in the storage unit 220.

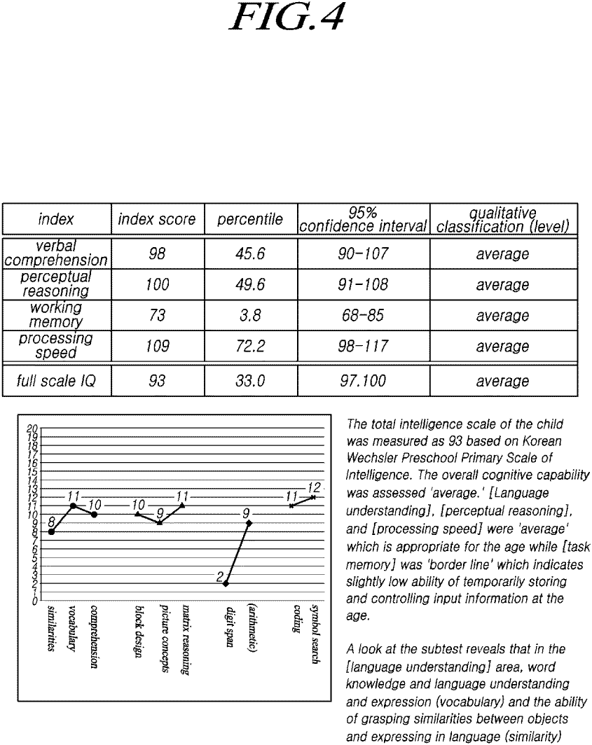

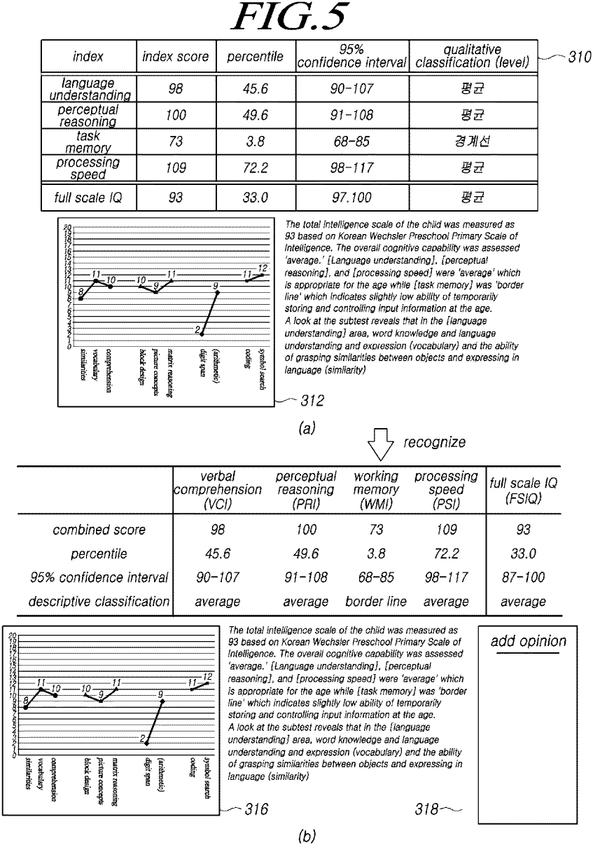

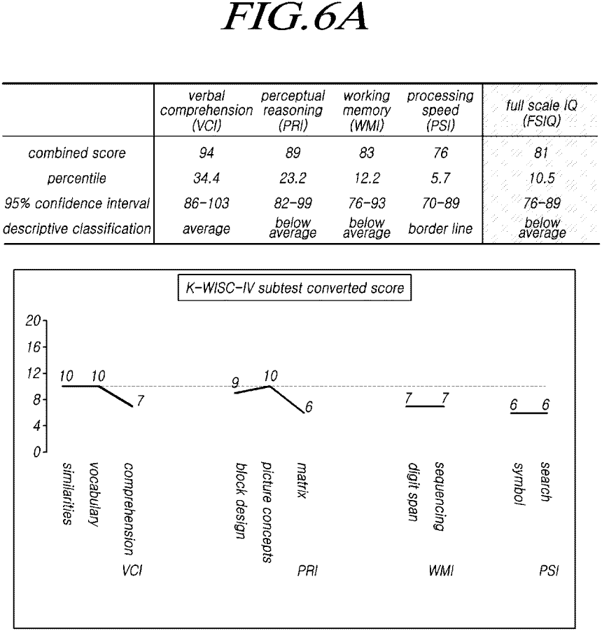

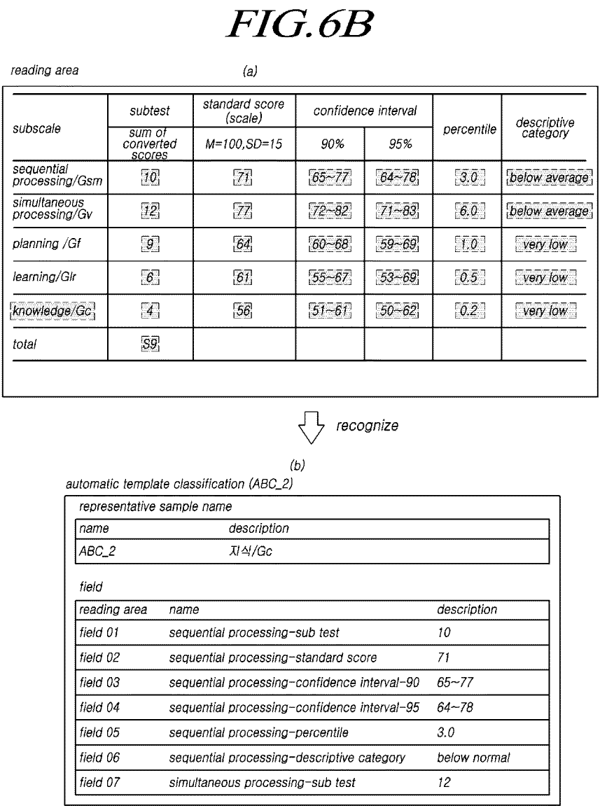

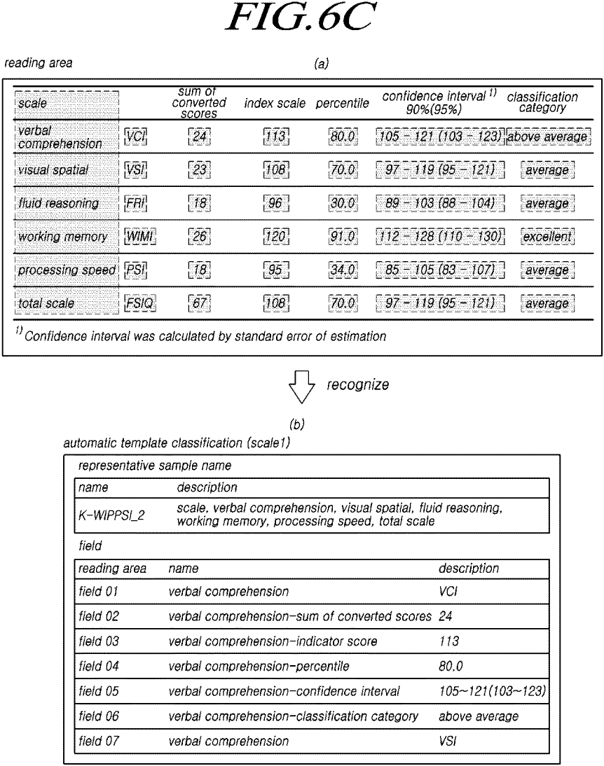

FIG. 4 illustrates an example of image data. FIG. 5 is a view illustrating a process of recognizing image information and text information from the image data of FIG. 4 and separating and storing them in a predetermined data format by the image recognition unit and the OCR unit of FIG. 3A. FIG. 6A shows an example of a test result newly stored.

Referring to FIG. 4, image data is image data that is captured through an input device, e.g., a camera and may be existing test data that evaluates the test results of the child with developmental disabilities, e.g., language understanding, perceptual reasoning, task memory, and processing speed and qualitatively classifies (level) them.

Referring to FIG. 5, the OCR unit 242 may also recognize text information 310 from the image data a input using deep learning technology which uses the artificial neural network or general character recognition technology, grasp the meaning of the test information 310, and divide the personal health record according to the type of each piece of information according to the predetermined data format 314.

Specifically, the input image data (FIG. 5(a)) is composed in a specific data format in which language understanding, perceptual reasoning, task memory, and processing speed are arranged vertically, and index score, percentile, 95% confidence interval, and qualitative classification (level) for each item are arranged horizontally.

The OCR unit 242 may recognize the text information 310 from the input image data ((a) of FIG. 5), grasp the meaning of the text information 310, and store it, in the predetermined data format 314 in which language understanding, perceptual reasoning, task memory, and processing speed are arranged horizontally, and index score, percentile, 95% confidence interval, and qualitative classification (level) for each item are arranged vertically, in the storage unit 220.

The image recognition unit 241 may recognize the image information 312 from the input image data ((a) of FIG. 5) using deep learning technology which uses the artificial neural network or general image recognition technology, to grasp the meaning of the recognized image information, divide the recognized image information according to a predetermined data format 316 depending on the type of the recognized image information and store them in the storage unit 220.

As shown in (b) of FIG. 5, the controller 240 may provide a screen (e.g., an item to add opinion) for inputting, through another server or a terminal, the item 318, which is not recognized by the OCR unit 242 and the image recognition unit 241 among the items of the data format of the personal health record stored in the storage unit 220, through the communication unit 210 or the output unit 250.

The controller 240 may provide a screen for identifying whether the image information and text information recognized by the image recognition unit 241 and the OCR unit 242 are accurate to another server or terminal through the communication unit 210 or the output unit 250 shown in FIG. 6A.

FIGS. 6B and 6C illustrate processes of automatically classifying, in a template, the image information and text information recognized by the image recognition unit 241 and the OCR unit 242 and storing them in the storage unit 220 by field.

Referring to FIGS. 6B and 6C, the controller 240 may automatically classify, by field, and datafication, the reading area of the text information and image information recognized by the OCR unit 242 and the image recognition unit 241 after created or entered in different data entry schemes in several organizations or hospitals.

As described above, the output unit 250 outputs the automatic template classification result. Resultantly, the server 200 may unify the units of data entered in different data entry schemes in several organizations or hospitals compatible, creating them into a unified template of data.

Referring back to FIG. 3A, the controller 240 may further include an artificial intelligence chatbot unit 243 with a chatbot function capable of chatting with at least one or more of a child with developmental disabilities, a play therapist, medical staff, and a child caregiver.

The artificial intelligence chatbot unit 243 may chat with at least one or two of the child with developmental disabilities, play therapist, medical staff, and child caregiver with reference to the personal health record including the patient information, clinical information and change trend information according to treatment stored in the storage unit 220.

The artificial intelligence chatbot unit 243 may include an artificial intelligence chatbot that is currently developing or to be developed in the future using artificial intelligence technology. The artificial intelligence chatbot unit 243 may answer the questions from at least one or two of the child with developmental disabilities, play therapist, medical staff, and child caregiver with reference to the personal health record including the patient information, clinical information and change trend information according to treatment stored in the storage unit 220.

The controller 240 may further include an abnormality detection unit 244 that detects the problematic behavior/medication status of children with developmental disabilities with reference to the personal health record including the patient information, clinical information and change trend information according to treatment stored in the storage unit 220.

When the abnormality detection unit 244 detects a problematic behavior/medication status of the child with developmental disabilities exceeding the normal range, the communication unit 210 may transmit an automatic alarm to another server or terminal through a network. The terminal may be the above-described patient terminal 110 or medical staff terminal 120. The other server may be a server linked with the server 200 that manages personal health records or a server linked with an emergency medical system, such as a medical institution or a public institution.

When transmitting an automatic alarm, the abnormality detection unit 244 stores the problematic behavior/medication status, the date and time of occurrence of the abnormal symptom, the accumulated number of abnormal occurrences, and if correlation with other information is found, the correlation, as the personal health record.

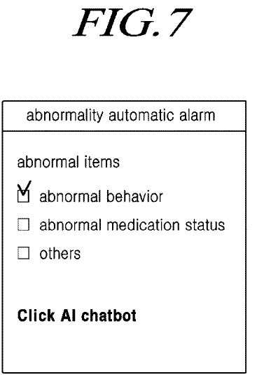

FIG. 7 illustrates an example of an automatic alarm message. As shown in FIG. 7, this automatic alarm message may also provide URL information that may be connected to the artificial intelligence chatbot performed by the above-described artificial intelligence chatbot unit 243 or information that may be connected or linked with other medical staff.

The controller 240 may include a medication management unit 245 that describes medication information about the medication provided during treatment management in relation to the personal health record including the patient information, clinical information and change trend information according to treatment stored in the storage unit 220. The output unit 250 may display the medication information described by the medication management unit 245.

The medication management unit 245 provides information such as general information about medication, medication guidance, recommended health food, medication guidance, and nutrient deficiency information. In particular, the medication management unit 245 does not provide only general medication information, but provides medication information in relation to the personal health records stored in the storage unit 220.

For example, when an automatic alarm for an abnormal medication status for a specific medication is provided by the above-described abnormality detection unit 244, medication information is provided in relation to the personal health record for the abnormal medication status stored in the storage unit 220. In other words, the medication management unit 245 may provide medication information including information indicating an alternative medication that exhibits the same efficacy as a specific medication that causes an abnormal medication condition but reflects the condition of a child with a specific developmental disability.

The medication management unit 245 may reflect the medication compliance, the % change in problematic behavior, % of daily task fulfillment, and the change in intelligence quotient of the same test result performed on different days, and if this condition persists, display a graph illuminating the level of developmental disability, the child’s emotional status and parental stress is changed through the output unit 250.

The controller 240 may further include a behavior correction unit 246 that automatically matches the diagnosis input by the medical staff or the problematic behavior input by the caregiver and the temperament input by the caregiver and presents a necessary task through the output unit 250.

The behavior correction unit 246 may provide social skills enhancement and behavior correction/reinforcement programs for children with developmental disabilities in an interesting animation format.

The artificial intelligence chatbot unit 2430 may cooperate with at least one of the abnormality detection unit 244, the medication management unit 245, and behavior correction unit 246 and refer to the personal health records added by them to answer the questions from at least one of the child with developmental disabilities, play therapist, medical staff, or child caregiver.

The above-described server 200 for integratedly managing personal health records allows an individual to integratedly manage treatment records necessary for children with developmental disabilities, such as physical therapy/sensory integration/cognitive therapy/language therapy, while enabling the transfer of accurate information to the hospital medical staff.

Further, the server 200 may retain the patient’s data as encrypted data by applying personal health information protection technology, thereby allowing only the patient to check the test records.

Further, the server 200 may provide each individual information, correlation, and tendency of diagnostic data, medication compliance, and app usage information by tagging professional data including the patient’s personal health record to determine the child’s condition from various angles, thus contributing to the enhancement of the sensitivity/specificity of diagnosis.

Further, the server 200 may secure medical data including a doctor’s expert opinion in forming an artificial intelligence algorithm, thereby greatly contributing to the development of the medical artificial intelligence field.

FIG. 3B is a view illustrating a configuration of another example of a controller of the server of FIG. 2A.

Referring to FIG. 3B, the controller 240 may include the image recognition unit 241 described with reference to FIG. 3A and an optical character recognition (OCR) unit 242.

The controller 240 may further include a data conversion unit 243b and an automatic translation unit 244b.

The data conversion unit 243b may integratedly recognize the test results between hospitals, which are created in different units and expression schemes, even for the same test. The data conversion unit 243b may make compatible and integratedly recognize the results of the same test executed in several development centers as well as from hospitals, particularly when the tests are made for children with developmental disabilities.

As described above, even for tests used worldwide, each test organization differently writes test results. The data conversion unit 243b may convert the test results into, e.g., a standard score (e.g., IQ, t-score, or z-score), percentile (%ile), high/medium/low, or excellent/poor. That is, the data conversion unit 243b may recognize each test result and convert it into a specific expression scheme.

As described above, the OCR unit 242 recognizes the text information 310 from the input image data (FIG. 5(a)) and grasps the meaning of the text information 310. Meanwhile, the input unit 230 may input the personal health record including patient information and clinical information, as digital data.

The data conversion unit 243b may convert the personal health information recognized by the OCR unit 242 or input through the input unit 230, e.g., each test result, in a specific expression scheme, e.g., standard score (e.g., IQ, t-score, or z-score), percentile (%ile), high/medium/low, or excellent/poor.

Personal health information may be create in various languages, such as English, Chinese, and Japanese as well as Korean. The automatic translation unit 244b may perform the task of automatically translating the personal health information in various languages into a specific language.

For example, if the personal health information input through the input unit 230 or recognized by the OCR unit 242 includes content created in different languages, the automatic translation unit 244b may automatically translate it into a specific language, e.g., Korean. The output unit 250 may display the automatically translated content in a specific language, e.g., Korean only, or in Korean along with the original language.

As another example, when the entire personal health information recognized by the OCR unit 242 or input through the input unit 230 is configured in a language other than Korean, the automatic translation unit 244b may automatically translate the entire personal health information into Korean. As described above, the output unit 250 may display the whole of the automatically translated personal health information in Korean or may display specific words or specific phrases in Korean and the original language both.

The automatic translation unit 244b may automatically translate sentences, paragraphs, or words using an artificial intelligence algorithm or a specific translation algorithm, e.g., a Markov algorithm.

As described above, when automatically classifying and creating into data the reading area of the text information and the image information recognized by the OCR unit 242 and the image recognition unit 241, by the field of the template, the controller 240 may perform such automatic classification and creation into data by the field of the template by referring to the automatically translated content of the automatic translation unit 244b.

Meanwhile, when converting the personal health information into specific units or a specific expression scheme, the data conversion unit 243b may perform such conversion of the personal health information into the specific units or specific expression scheme by referring to the content automatically translated by the automatic translation unit 244b.

The above-described server 200 for integratedly managing personal health records as global big data converts the personal health information into specific unis or a specific expression scheme by the data conversion unit 243b or automatically translates the whole or part of the personal health information into various languages and automatically classifies them by the field of the template to thereby create into data by the automatic translation unit 244b. Thus, the personal health information created in various languages in hospitals or development centers in various countries may be created into global big data.

As such, since the server 200 creates the personal health information created in various languages in hospitals or development centers in various countries into global big data, when the image input unit 241 or the OCR unit 242 uses artificial intelligence, the amount of learning data may be increased, so that the learning performance of the artificial intelligence may be enhanced.

The server for integratedly managing personal health records according to an embodiment has been described above with reference to FIGS. 1 to 7. Hereinafter, a method for integratedly managing personal health records according to another embodiment is described with reference to the drawings. This method may be performed by the above-described server for integratedly managing personal health records or by another server or another terminal.

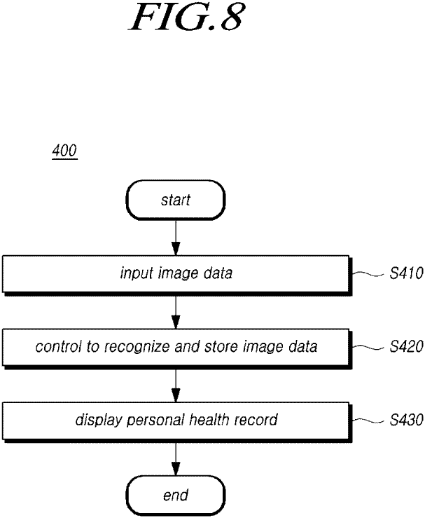

FIG. 8 is a flowchart illustrating a method for integratedly managing personal health records for children with developmental disabilities according to another embodiment.

Referring to FIG. 8, a method 400 for integratedly managing personal health records, including clinical information for children with developmental disabilities and change trend information according to treatment includes the step S410 of receiving image data, the step S420 of controlling to recognize one of image information and text information from the received image data and divide and store one of the recognized image information and text information into patient information and clinical information and change trend information according to treatment in a predetermined data format, and the step S430 of displaying a personal health record including the stored patient information, clinical information, and change trend information according to treatment according to a request.

The image data may be one of the image data of test data prepared in a specific data format, prescription image data, and a medical report.

The controlling step S420 may i) recognize image information from one of the image data of test data, prescription image data, and medical report, ii) recognize text information from one of the image data of test data, prescription image data, and medical report, and iii) divide and store one of the image information and text information recognized from one of the image data of test data, prescription image data, and medical report created in a specific data format into patient information and clinical information and change trend information according to treatment according to a predetermined data format.

As described with reference to FIGS. 4 and 5, the controlling step S420 may recognize text information 310 from the image data a input using deep learning technology which uses the artificial neural network or general character recognition technology, grasp the meaning of the test information 310, and divide the personal health record according to the type of each piece of information according to the predetermined data format 314.

Specifically, the input image data (a) is composed in a specific data format in which language understanding, perceptual reasoning, task memory, and processing speed are arranged vertically, and index score, percentile, 95% confidence interval, and qualitative classification (level) for each item are arranged horizontally.

The controlling step S420 may recognize the text information 310 from the input image data (a), grasp the meaning of the text information 310, and store it, in the predetermined data format 314 in which language understanding, perceptual reasoning, task memory, and processing speed are arranged horizontally, and index score, percentile, 95% confidence interval, and qualitative classification (level) for each item are arranged vertically, in the storage unit 220.

The controlling step S420 may recognize the image information 312 from the input image data (a) using deep learning technology which uses the artificial neural network or general image recognition technology, grasp the meaning of the recognized image information, divide the recognized image information according to a predetermined data format 316 depending on the type of the recognized image information and store them.

As shown in (b) of FIG. 5, the controlling step S420 may provide a screen for entering the item 318, which is not recognized among the items of the data format of the stored personal health record, through another server or terminal.

The controlling step S420 may provide a screen for identifying whether the recognized image information and text information is accurate to another server or terminal.

The controlling step S420 may perform a chatbot function capable of chatting with at least one or more of a child with developmental disabilities, a play therapist, medical staff, and a child caregiver.

The chatbot function may chat, in a question-and-answer type, with at least one or two of the child with developmental disabilities, play therapist, medical staff, and child caregiver with reference to the stored personal health record including the patient information, clinical information and change trend information according to treatment.

The controlling step S420 may detect the problematic behavior/medication status of children with developmental disabilities with reference to the stored personal health record including the patient information, clinical information and change trend information according to treatment and, when detecting a problematic behavior/medication status of the child with developmental disabilities, can transmit an automatic alarm through the network 130.

As described with reference to FIG. 7, the automatic alarm message may also provide URL information that may be connected to the artificial intelligence chatbot or information that may be connected or linked with other medical staff.

The controlling step S420 may describe medication information about the medication provided during treatment management in relation to the stored personal health record including the patient information, clinical information and change trend information according to treatment and control to display the medication information.

For example, when an automatic alarm for an abnormal medication status for a specific medication is provided, there may be provided medication information including information indicating an alternative medication that exhibits the same efficacy as a specific medication that causes an abnormal medication condition but reflects the condition of a child with a specific developmental disability.

The controlling step S420 may reflect the medication compliance, the % change in problematic behavior, % of daily task fulfillment, and the change in intelligence quotient of the same test result performed on different days, and if this condition persists, display a graph showing the level of developmental disability, the child’s emotional status and parental stress is changed.

The controlling step S420 may automatically match the diagnosis input by the medical staff or the problematic behavior input by the caregiver and the temperament input by the caregiver and presents a necessary task. For example, there may be provided social skills enhancement and behavior correction/reinforcement programs for children with developmental disabilities in an interesting animation format.

The controlling step S420 may analyze the personal health record profile of the child, matches the categories of users with similar profiles to the corresponding interpreted information (psychological assessment information database), searches for customized information based thereon, and then provide per-profile customized information which is customized information for the caregiver. The controlling step S420 may analyze the personal health record stored by the caregiver, extract the child’s developmental information, and provide service or customized treatment program recommendation information that may recommend useful developmental stimuli according to thereto.

The controlling step S420 may recommend an evidence-based treatment module when a clinically significant functional decline is observed in the developmental assessment. For example, the controlling step S420 may recommend evidence-based treatment modules according to developmental and emotional states through artificial intelligence. Evidence-based treatment modules refer to treatments whose effects have been found through well-designed studies, and may help establish a more accurate treatment plan.

The controlling step S420 may build big data through matching between the knowledge base (clinician’s interpretation) and the database of the storage unit 420 and form and enhance a specific algorithm.

The controlling step S420 may integratedly recognize test results of hospitals, which are created BCL, r Scalesventory ; children’ in different units and expression schemes even for the same test. The controlling step S420 may make compatible and integratedly recognize the results of the same test executed in several development centers as well as from hospitals, particularly when the tests are made for children with developmental disabilities.

As described above with reference to FIGS. 2B and 2C, when displaying a description of each test result, the displaying step S430 may attach tags to difficult terms or items that a user might be curious about the test. When the user selects one of the tags, the output unit 250 may blur the test result and provide a detailed description of the esoteric term or questions about the test with the selected tag, linked to an Internet search information or medical terminology dictionary.

Further, as described above with reference to FIG. 2D, the displaying step S430 may output a test record to be able to observe follow-ups and changes for the individual’s annual test record. Further, the displaying step S430 may output follow-up observation results of changes by indicator for each subtest and changes by developmental period for one test, thus output customized information.

Further, as described above in connection with FIG. 2D, the displaying step S430 may output changes for each intelligence test indicator and AI-based customized information for one test. For example, the controlling step S420 may control the displaying step S430 to output user-customized information through profile analysis of various information, such as profile analysis of intelligence test development information and/or profile analysis of temperament and personality information, and profile analysis of emotional state information.

Thus, the method 400 for integratedly managing the personal health record may provide user-customized information through profile analysis of each test.

Further, as described above in connection with FIG. 2E, the displaying step S430 may convert the comparable top items into a unified unit value (percentile %), integrate them as a “comprehensive development report”, and visualize it as a radial graph. Thus, the user or medical staff may assess the overall development status at a glance.

The displaying step S430 may display customized welfare information according to expected diagnosis and disabilities according to the output of the comprehensive development report. For example, the displaying step S430 may provide legal information, such as the Welfare Act for Persons with Disabilities from the government, public institutions, and private organizations, and support information, such as national support/local government support or private organization support. The user may utilize customized welfare information, such as the provided legal information or support information.

FIGS. 6B and 6C illustrate processes of automatically classifying, in a template, the image information and text information recognized by the image recognition unit 241 and the OCR unit 242 and storing them by field.

As described above with reference to FIGS. 6B and 6C, the controlling step S420 may automatically classify, by field, and create into data, the reading area of the text information and image information recognized after created or entered in different data entry schemes in several organizations or hospitals.

As described above, the outputting step S430 outputs the automatic template classification result.

Thus, the method 400 for integratedly managing personal health records may make the data entered in different data entry schemes in several organizations or hospitals compatible, creating them into a unified data template.

The above-described method 400 for integratedly managing personal health records allows an individual to integratedly manage treatment records necessary for children with developmental disabilities, such as physical therapy/sensory integration/cognitive therapy/language therapy while enabling transfer of accurate information to the hospital medical staff.

Further, the method 400 may retain the patient’s data as encrypted data by applying personal health information protection technology, thereby allowing only the patient to check the test records.

Further, the method 400 may provide each individual information, correlation, and tendency of diagnostic data, medication compliance, and app usage information by tagging professional data including the patient’s personal health record to determine the child’s condition from various angles, thus contributing to the enhancement of the sensitivity/specificity of diagnosis.

Further, the server 200 may secure medical data including a doctor’s expert opinion in forming an artificial intelligence algorithm, thereby greatly contributing to the development of the medical artificial intelligence field.

Referring back to FIG. 8, the controlling step S420 may integratedly recognize test results of hospitals, which are created in different units and expression schemes even for the same test. The controlling step S420 may make compatible and integratedly recognize the results of the same test executed in several development centers as well as from hospitals, particularly when the tests are made for children with developmental disabilities.

As described above, even for tests used worldwide, each test organization differently writes test results. The controlling step S420 may convert the test results into, e.g., a standard score (e.g., IQ, t-score, or z-score), percentile (%ile), high/medium/low, or excellent/poor. That is, the controlling step S420 may recognize each test result and convert it into a specific expression scheme.

The controlling step S420 may convert the personal health information, which is recognized or input, e.g., each test result, in a specific expression scheme, e.g., standard score (e.g., IQ, t-score, or z-score), percentile (%ile), high/medium/low, or excellent/poor.

Personal health information may be created in various languages, such as English, Chinese, and Japanese as well as Korean. The controlling step S420 may perform the task of automatically translating the personal health information in various languages into a specific language.

For example, if the recognized or input personal health information includes content created in different languages, the controlling step S420 may automatically translate the content into a specific language, e.g., Korean. The outputting step S430 may display the automatically translated content in a specific language, e.g., Korean only, or in Korean along with the original language.

For another example, when the entire personal health information recognized or input is configured in a language other than Korean, the controlling step S420 may automatically translate the entire personal health information into Korean. As described above, the outputting step S430 may display the whole of the automatically translated personal health information in Korean or may display specific words or specific phrases in Korean and the original language both.

The controlling step S420 may automatically translate sentences, paragraphs, or words using an artificial intelligence algorithm or a specific translation algorithm, e.g., a Markov algorithm.

As described above, when automatically classifying and creating into data the reading area of the recognized text information and image information by the field of the template, the controlling step S420 may perform such automatic classification and creation into data by the field of the template by referring to the automatically translated content.

Meanwhile, when converting the personal health information into specific units or a specific expression scheme, the controlling step S420 may perform such conversion of the personal health information into the specific units or specific expression scheme by referring to the automatically translated content.

The above-described server 200 for integratedly managing personal health records as global big data converts the personal health information into specific units or a specific expression scheme or automatically translates the whole or part of the personal health information into various languages and automatically classifies them by the field of the template to thereby create into data. Thus, the personal health information created in various languages in hospitals or development centers in various countries may be created into global big data.

As such, since the method 400 for integratedly managing personal health records creates the personal health information created in various languages in hospitals or development centers in various countries into global big data, it is possible to increase the amount of learning data when using artificial intelligence and accordingly enhance the learning performance of the artificial intelligence.

Another embodiment provides a computer program stored in a computer recording medium, performing the above-described method for integratedly managing personal health records for children. Further, another embodiment provides a computer-readable recording medium storing a program for realizing the above-described method for integratedly managing personal health records for children with developmental disabilities.

The program stored in the recording medium to realize the above-described method for integratedly managing personal health records for children with developmental disabilities may be read and installed on the computer and executed to perform the above-described steps.

As such, for the computer to read the program recorded on the recording medium and execute the implemented functions with the program, the above-described program may include code coded in a computer language, such as C, C++, JAVA, or machine language, which the processor (CPU) of the computer may read through a computer device interface.

Such code may include a function code related to a function defining the above-described functions or may include an execution procedure-related control code necessary for the processor of the computer to execute the above-described functions according to a predetermined procedure.

Further, the code may include additional information necessary for the computer’s processor to execute the above-described functions or memory reference-related code as to the position (or address) in the internal or external memory of the computer the media should reference.

Further, when the processor of the computer needs to communicate with, e.g., another computer or a server at a remote site to execute the above-described functions, the code may further include communication-related code as to how the processor of the computer should communicate with the remote computer or server using the communication module of the computer and what information or media should be transmitted/received upon communication.

The above-described computer-readable recording medium may include, e.g., ROMs, RAMs, CD-ROMs, magnetic tapes, floppy disks, or optical data storage devices, or may also include carrier wave-type implementations (e.g., transmissions through the Internet).

Further, the computer-readable recording medium may be distributed to computer systems connected via a network, and computer-readable codes may be stored and executed in a distributed manner.

The functional programs for implementing the present invention and code and code segments related thereto may easily be inferred or changed by programmers of the technical field to which the present invention pertains, considering, e.g., the system environments of the computer reading and executing the program.

As described in connection with FIG. 5, the method for integratedly managing personal health records for children with developmental disabilities based on big data and artificial intelligence may be implemented in the form of a recording medium or computer-readable medium containing computer-executable instructions or commands, such as an application or program module executable on a computer. The computer-readable medium may be an available medium that is accessible by a computer. The computer-readable storage medium may include a volatile medium, a non-volatile medium, a separable medium, and/or an inseparable medium. The computer-readable medium may include a computer storage medium. The computer storage medium may include a volatile medium, a non-volatile medium, a separable medium, and/or an inseparable medium that is implemented in any method or scheme to store computer-readable commands, data architecture, program modules, or other data or information.

The above-described method for integratedly managing personal health records for children with developmental disabilities according to an embodiment of the present invention and the method for integratedly managing personal health records for children with developmental disabilities according to another embodiment may be executed by an application installed on a terminal, including a platform equipped in the terminal or a program included in the operating system of the terminal), or may be executed by an application (or program) installed by the user on a master terminal via an application providing server, such as a web server, associated with the service or method, an application, or an application store server. According to an embodiment, the above-described method for integratedly managing personal health records for children with developmental disabilities may be implemented in an application or program installed as default on the terminal or installed directly by the user and may be recorded in a recording medium or storage medium readable by a terminal or computer.

Although embodiments of the present invention have been described with reference to the accompanying drawings, it will be appreciated by medical staff and clinicians who may not have strong skills in this area that the present disclosure may be implemented in other various specific forms without changing the essence or technical spirit of the present disclosure. Thus, it should be noted that the above-described embodiments are provided as examples and should not be interpreted as limiting. Each component may be separated into two or more units or modules to perform its function(s) or operation(s), and two or more components may be integrated into a single unit or module to perform their functions or operations.

It should be noted that the scope of the present invention is defined by the appended claims rather than the described description of the embodiments and include all modifications or changes made to the claims or equivalents of the claims.

1-21. (canceled)

22. A server for integratedly managing personal health records for children with developmental disabilities, comprising:
a communication unit communicating with another server or a terminal through a network and receiving image data from the other server or the terminal;
a storage unit storing a personal health record including patient information and clinical information;
an input unit receiving image data;
a controller recognizing one of image information and text information from the received image data, dividing and storing, in the storage unit, one of the recognized image information and the text information into the patient information and the clinical information in a predetermined data format; and
an output unit displaying the personal health record including the patient information and the clinical information stored in the storage unit, according to a request.

23. The server of claim 22, wherein the image data is one of the image data of test data prepared in a specific data format, prescription image data, and a medical report,
wherein the controller includes an image recognition unit recognizing the image information from one of the image data of test data, prescription image data, and medical report and an optical character recognition (OCR) unit recognizing the text information from one of the image data of test data, prescription image data, and medical report, and
wherein the controller divides one of the image information and text information recognized from one of the image data of test data, prescription image data, and medical report created in the specific data format into patient information and clinical information according to a predetermined data format and stores them in the storage unit.

24. The server of claim 23, wherein the controller automatically classifies and creates into data a reading area of image information and text information created or input in different data input schemes and then recognized by the image recognition unit and the OCR unit, by the field of a template.

25. The server of claim 22, wherein the controller further includes an AI chatbot unit that performs a chatbot function capable of chatting with at least one or two of the child with developmental disabilities, a play therapist, medical staff, and a child caregiver, and
wherein the artificial intelligence chatbot unit chats with at least one or two of the child with developmental disabilities, the play therapist, the medical staff, and the child caregiver with reference to the personal health record including the patient information, clinical information and change trend information according to treatment stored in the storage unit.

26. The server of claim 22, wherein the controller further includes an abnormality detection unit detecting a problematic behavior/medication status of the child with developmental disability with reference to the personal health record including the patient information and the clinical information stored in the storage unit, and
wherein when the abnormality detection unit detects the problematic behavior/medication status of the child with developmental disability, the communication unit transmits an automatic alarm to the other server or the terminal through the network.

27. The server of claim 22, wherein the controller further includes a medication management unit describing medication information about a medication provided during treatment management in relation to the personal health record including the patient information and the clinical information stored in the storage unit, and
wherein the output unit displays the medication information described by the medication management unit.

28. The server of claim 27, wherein the medication management unit reflects a medication compliance, a % change in problematic behavior, % of daily task fulfillment, and a change in intelligence quotient of the same test result performed on different days, and if the condition persists, displays, through the output unit, a graph showing the level of developmental disability, the child’s emotional status, and parental stress is changed.

29. The server of claim 22, wherein the controller further includes a behavior correction unit automatically matching a diagnosis input by medical staff or a problematic behavior input by a caregiver and a temperament input by the caregiver, presenting a necessary task through the output unit.

30. The server of claim 24, wherein the controller further includes a data conversion unit converting inter-hospital test results created in different units and expression schemes even for the same test into a specific unit or specific expression scheme.

31. The server of claim 24, wherein the controller further includes an automatic translation unit automatically translating contents respectively created in different languages in a whole or part of the personal health information recognized by the OCR unit or input through the input unit into a specific language.

32. A method for integratedly managing personal health records for children with developmental disabilities, the method comprising:
receiving image data;
recognizing one of image information and text information from the received image data and controls to divide and store one of the recognized image information and the text information into the patient information and the clinical information in a predetermined data format; and
displaying the personal health record including the stored patient information, the clinical information, and the change trend information according to treatment according to a request.

33. The method of claim 32, wherein the image data is one of the image data of test data prepared in a specific data format, prescription image data, and a medical report,
wherein the controlling step i) recognizes the image information from one of the image data of test data, prescription image data, and medical report, ii) recognizes the text information from one of the image data of test data, prescription image data, and medical report, and iii) divides and stores one of the image information and text information recognized from one of the image data of test data, prescription image data, and medical report created in a specific data format into the patient information and the clinical information according to a predetermined data format.

34. The method of claim 32, wherein the controlling step automatically classifies and creates into data a reading area of image information and text information created or input in different data input schemes and then recognized, by the field of a template.

35. The method of claim 32, wherein the controlling step performs a chatbot function capable of chatting with at least one or two of a child with developmental disabilities, a play therapist, medical staff, and a child caregiver, and wherein the chatbot function chats with at least one or two of the child with developmental disabilities, the play therapist, the medical staff, and the child caregiver with reference to the stored personal health record including the patient information, clinical information and change trend information according to treatment.

36. The method of claim 32, wherein the controlling step detects a problematic behavior/medication status of the child with developmental disability with reference to the stored personal health record including the patient information, clinical information and change trend information according to treatment and, when the problematic behavior/medication status of the child with developmental disabilities is detected, controls to transmit an automatic alarm through a network.

37. The method of claim 32, wherein the controlling step describes medication information about a medication provided during treatment management in relation to the personal health record including the patient information and the clinical information stored in the storage unit and controls to display the medication information.

38. The method of claim 32, wherein the controlling step reflects a medication compliance, a % change in problematic behavior, % of daily task fulfillment, and a change in intelligence quotient of the same test result performed on different days, and if the condition persists, displays a graph showing the level of developmental disability, the child’s emotional status, and parental stress is changed.

39. The method of claim 32, wherein the controlling step automatically matches a diagnosis input by medical staff or a problematic behavior input by a caregiver and a temperament input by the caregiver and presents a necessary task.

40. The method of claim 34, wherein the controlling step converts inter-hospital test results created in different units and expression schemes even for the same test into a specific unit or specific expression scheme.

41. The method of claim 34, wherein the controlling step recognizes the text information or automatically translates contents respectively created in different languages in a whole or part of the personal health information into a specific language.

42. A computer-readable recording medium storing a program for implementing the method for integratedly managing personal health records for children with developmental disabilities of claim 32.
